(19) United States
(12) Patent Application Publication
(10) Pub. No.: US 2018/0333379 A1
(43) Pub. Date: Nov. 22, 2018

(54) CITRATE DISSOLUTION OF BETA-2-MICROGLOBULIN AND AMYLOID BETA PEPTIDE (1-40) AGGREGATES

(71) Applicant: Joel Goldberg, Hillsborough, NC (US)

(72) Inventor: Joel Steven Goldberg, Hillsborough, NC (US)

(21) Appl. No.: 16/027,324

(22) Filed: Jul. 4, 2018

(51) Int. Cl. A61K 31/194; A61K 9/00; A61P 25/28

(52) U.S. Cl. CPC A61K 31/194; A61P 25/28; A61K 9/0019; A61K 9/0053

(57) ABSTRACT

Amyloid and amyloid-like proteins are found in many diseases including Alzheimer's disease, type 2 diabetes, rheumatoid arthritis, spongiform encephalopathies, and chronic renal failure. Citrate in concentrations of 0.1 mM to 10 mM can dissolve amyloid-like protein aggregates of β-2 microglobulin and amyloid β peptide₍₁₋₄₀₎ presumably by chelating calcium. Citrate can be absorbed from the gastrointestinal tract and crosses the blood brain barrier where the concentration of citrate is two to three times the plasma concentration. Citrate administered orally or parenterally may be an effective low-risk treatment and a prevention for diseases characterized by amyloid-like deposits. Clinical trials are needed.

### CROSS-REFERENCES TO RELATED APPLICATIONS

None

### FEDERALLY FUNDED RESEARCH

None

### BACKGROUND OF THE INVENTION

Amyloids are a group of proteins characterized by parallel beta sheets. When secondary protein structure changes from helices to parallel sheets, amyloid proteins may be formed. Amyloid-like deposits are associated with many diseases including Alzheimer's disease, type 2 diabetes, spongiform encephalopathies, rheumatoid arthritis, and chronic renal failure.

Calcium, along with other metal ions such as copper, zinc, and aluminum, have been shown to produce amyloid-like aggregates when incubated with β-2-microglobulin or β-amyloid peptide₍₁₋₄₀₎. (Wang et al., 2014) (Kumar, Sharma, Arora, Raje, & Guptasarma, 2014) This spontaneous reaction occurs at 37° C. over a few weeks. These aggregates fluoresce when stained with thioflavin T, a common stain for amyloid. Amyloid from β-2-microglobulin aggregates is believed to cause dialysis related amyloidosis and elevated levels of β-2-microglobulin, which are found in multiple myeloma, occult malignancies, and rheumatoid diseases. (Argyropoulos et al., 2017) Amyloid deposits from β-amyloid peptide₍₁₋₄₀₎ are found in the plaques of patients with Alzheimer's disease. When ethylenediamine tetraacetic acid (EDTA) is incubated with calcium treated β-2-microglobulin aggregates, the aggregates can be dissolved presumably by chelation of calcium.(Kumar et al., 2014)

Citrate has been shown to inhibit formation of amyloid-like aggregates when incubated with β-amyloid peptide₍₁₋₄₀₎. (Park, Kim, Son, & Yang, 2009) The mechanism of action was believed to be secondary to hydrophilic/electrostatic action of citrate to reduced hydrophobicity, but may be secondary to chelation of calcium by citrate. Furthermore, citrate was shown to protect neurons from the apoptotic effects of β-amyloid peptide₍₁₋₄₀₎. (Park et al., 2009)

Citrate is commonly used to anticoagulate blood by chelating calcium. This chelation reaction has also been shown to soften cellulose in grasses, permitting manual extraction of protein from grasses, presumably by chelating calcium which binds to pectin. (Goldberg, 2016)

Previous work has shown that citrate in the form of sodium citrate buffer in concentrations of 280-310 milliosmoles per liter at pH of 6-8 had minimal effect on the weight of ligamentum flavum, dura mater, and spinal cord in an animal preparation, but decreased vertebral bone weight and could be a treatment for spinal stenosis. (Goldberg, 2017)

Citrate is readily absorbed from the human gastrointestinal tract and approximately 30% of plasma citrate is metabolized to bicarbonate in the liver.(Haerer, 1971) Citrate freely crosses the blood brain barrier, and the cerebral spinal fluid to plasma concentration of citrate is approximately two-three to one. Average plasma citrate concentration in normal volunteers is 1.67 mg/100 mg or 0.09 mM, and the average CSF concentration is 0.18 mM. In patients with cerebrovascular disorders the CSF citrate concentration can rise to 0.3 mM. (Haerer, 1971)

Citrate levels in human plasma are homeostatically regulated. The largest stores of citrate in humans are in bone. Parathyroid hormone and calcitonin are the major hormones responsible for citrate regulation. Increases in plasma vitamin D and plasma parathyroid hormone may increase plasma citrate levels. Also, renal clearance, and under specific conditions hepatic clearance are involved in citrate regulation.(Costello & Franklin, 2016)

Citrate can be administered orally or parenterally. Oral administration of citric acid, particularly from citrus fruits, and sodium citrate is common in the human diet. Parenteral absorption includes intravenous and intrathecal administration. Fentanyl citrate, with a citrate concentration of 0.09 mM, and hydromorphone, in a citric acid/ sodium citrate buffer with a citrate concentration of 0.017 mM, are commonly administered intrathecally in clinical practice.

In this invention, it was shown that citrate can dissolve, presumably by chelation of calcium, precipitated forms of amyloid-like aggregates of β-2-microglobulin and amyloid β peptide₍₁₋₄₀₎ at citrate concentrations approximating those found in the human plasma. Furthermore, it is postulated that citrate in vivo may decrease the formation of amyloid-like aggregates of β-2-macroglobulin and amyloid β peptide₍₁₋₄₀₎. Also, it was also shown that calcium did not precipitate aggregates of alpha synuclein, which is associated with Parkinson's disease, under similar conditions that amyloid-like aggregates of β-2-macroglobulin and amyloid β peptide₍₁₋₄₀₎ aggregates were formed.

### DESCRIPTION OF THE DRAWINGS

None

### DETAILED DESCRIPTION OF THE INVENTION

In the following experiments, the concentration of thioflavin T was thirty micromolar in phosphate buffered saline (PBS), and the staining time was five minutes.

In the following experiments, an unblended observer interpreted fluorescence. Interpretation of microscopic fluorescence intensity may be subject to bias. Staining times and washes were controlled.

1. Citrate Treatment of Beta-2-Microglobulin Aggregates

One milliliter of β-2-microglobulin (Lee Biosolutions, Maryland Heights, Mo.) at a concentration of 1 mg/0.5 ml were mixed with two milliliters of 10 mM calcium chloride in normal saline at 37° C. for three weeks, and a precipitate formed. The supernatant calcium chloride was aspirated, and and ten microliter aliquots of the precipitate were treated with 190 microliters of 0.01 mM ,0.1 mM, 1.0 mM, 10.0 mM,100 mM, and 1.0 M solutions of sodium citrate, respectively. After 2 week of citrate treatment the samples were centrifuged at 1500 rpm for five minutes. The precipitate was aspirated and stained with 10 microliters of thioflavin T, washed twice with phosphate bufferd saline (PBS), and each sample was observed under an epifluorescent microscope with B2 filter (excitation filter, 450-490 nm; barrier filter, 520 nm). (Table 1)

TABLE 1
Citrate treatment of β-2-microglobulin aggregates.

| Precipitated | | | Presence of |
|---|---|---|---|
| β-2 microglobulin | Sodium citrate | | fluorescent fibrils |
| Ten μl β-2 microglobulin | Zero | | Present |
| Ten μl β-2 microglobulin | 0.01 | mM | Present |
| Ten μl β-2 microglobulin | 0.1 | mM | Present |
| Ten μl β-2 microglobulin | 10 | mM | Absent |
| Ten μl β-2 microglobulin | 100 | mM | Absent |

| | | |
|---|---|---|
| Ten μl β-2 microglobulin | 1M | Present |

Negative controls included: 1) five microliters of thioflavin T 2) five microliters of non-aggregated β-2-microglobulin 3) five microliters of non-aggregated B-2microglobulin with ten microliters of thioflavin T. (Table 2)

TABLE 2
Controls for fluorescence studies of β-2-microglobulin

| | % |
|---|---|
| Sample | fluorescence |
| Five microliters of thioflavin T | Zero |
| Five microliters of β-2-microglobulin (non-aggregate) | Zero |
| Five microliters of β-2-microglobulin (non-aggregate) | Zero |
| with ten microliters of thioflavin T | |

2. Citrate Treatment of Amyloid β Peptide₍₁₋₄₀₎ Aggregates

One milligram of amyloid β peptide₍₁₋₄₀₎ (Abcam, Cambridge, United Kingdom) was dissolved in 400 microliters of 10 mM calcium chloride in PBS and incubated at 37° C. for three weeks. A precipitate formed and the sample was centrifuged at 1500 rpm for five minutes. Five microliter samples were incubated with 95 microliters of sodium citrate in PBS at concentrations of 10.0 mM, 1.0 mM, 0.1 mM, and 0.01 mM for 48 hours at 37° C. Then, five microliters of thioflavin T were added to each sample, and after five minutes, the samples were centrifuged at 1500 rpm for five minutes and washed twice with PBS. Aggregates in the micro centrifuge tubes were aspirated and observed under an epifluorescent microscope with B2 filter. (Table 3)

TABLE 3
Citrate treatment of amyloid β peptide ₍₁₋₄₀₎ aggregates

| Precipitated amyloid | | | Thioflavin T |
|---|---|---|---|
| β peptide₍₁₋₄₀₎ | Sodium citrate | | fluorescence |
| Five μl amyloid β peptide ₍₁₋₄₀₎ | Zero | | Present |
| Five μl amyloid β peptide ₍₁₋₄₀₎ | 10.00 | mM | Absent |
| Five μl amyloid β peptide ₍₁₋₄₀₎ | 1.00 | mM | Minimally present |
| Five μl amyloid β peptide ₍₁₋₄₀) | 0.10 | mM | Present |
| Five μl amyloid β peptide ₍₁₋₄₀₎ | 0.01 | mM | Present |

3. Calcium Chloride Treatment of Alpha Synuclein

Five hundred micrograms of alpha synuclein (rPeptide, LLC, Athens, Ga.) was dissolved in 400 microliters of 10 mM calcium chloride in PBS and incubated at 37° C. for four weeks. No precipitate formed and no precipitate was found after fifteen minutes of centrifugation at 1500 rpm.

### Discussion

β-2 microglobulin and amyloid β peptide₍₁₋₄₀₎ form spontaneous aggregates when incubated with calcium chloride at 37° C. that fluoresce when stained with thioflavin T. Preliminary data suggests that these aggregates dissolve and no longer fluoresce when incubated in various concentrations of sodium citrate. β-2 microglobulin aggregates are associated with amyloid diseases, and amyloid β peptide₍₁₋₄₀₎ aggregates are associated with plaques found in Alzheimer's disease. At the present time, there are no good therapies for the treatment of amyloid and Alzheimer's diseases. If plasma and CSF levels of citrate can be increased by oral or parenteral, including intrathecal, administration of citrate, this may prevent formation of β-2 microglobulin aggregates and amyloid β peptide₍₁₋₄₀₎ aggregates.

### Benefits to Society

Amyloid and Alzheimer's diseases produce a significant burden upon those afflicted with the illnesses and upon society, and there are no good preventative or curative therapies. In this invention, it was shown that citrate can dissolve recently formed β-2 microglobulin aggregates and amyloid β peptide₍₁₋₄₀₎ aggregates in vitro. Citrate in the form of sodium citrate or citric acid has a low toxicity and can be ingested as a citrus drink, such as lemonade or limeade, or incorporated into common recipes as sodium citrate. Large quantities of citrate acid can be pleasantly ingested in aqueous solution with lemon extract and a sweetener. Increases in plasma vitamin D and parathyroid hormone also promote hypercitricemia, and together with administration of citrate, may be beneficial to those suffering amyloid and Alzheimer's diseases. Hypercitricemia may also prevent the development of amyloid and Alzheimer's diseases in predisposed populations.

### REFERENCES

Argyropoulos, C. P., Chen, S. S., Ng, Y. H., Roumelioti, M. E., Shaffi, K., Singh, P. P., & Tzamaloukas, A. H. (2017). Rediscovering Beta-2 Microglobulin As a Biomarker across the Spectrum of Kidney Diseases. Front Med (Lausanne), 4, 73.

Costello, L. C., & Franklin, R. B. (2016). Plasma Citrate Homeostasis: How It Is

Regulated; And Its Physiological and Clinical Implications. An Important, But Neglected, Relationship in Medicine. HSOA J Hum Endocrinol, 1(1).

Goldberg, J. S. (2016). U.S. patent application Ser. No. 16/0270425 A1. Washington, D.C.: USPTO.

Goldberg, J. S. (2017). U.S. Pat. No. 9,616,040 B2. Washington, D.C.: USPTO

Haerer, A. F. (1971). Citrate and alpha-ketoglutarate in cerebrospinal fluid and blood. Neurology, 21(10), 1059-1065.

Kumar, S., Sharma, P., Arora, K., Raje, M., & Guptasarma, P. (2014). Calcium binding to beta-2-microglobulin at physiological pH drives the occurrence of conformational changes which cause the protein to precipitate into amorphous forms that subsequently transform into amyloid aggregates. PLoS One, 9(4).

Park, Y. H., Kim, Y. J., Son, I. H., & Yang, H. D. (2009). Inhibition of beta-amyloid(1-40) Peptide Aggregation and Neurotoxicity by Citrate. Korean J Physiol Pharmacol, 13(4), 273-279.

Wang, L., Hu, J., Zhao, Y., Lu, X., Zhang, Q., & Niu, Q. (2014). Effects of aluminium on beta-amyloid (1-42) and secretases (APP-cleaving enzymes) in rat brain. Neurochem Res, 39(7), 1338-1345.

Having described my invention, I claim:

1. A method to prevent and treat amyloid diseases in a human subject comprising parenteral or oral administration of citrate to said subject.

2. A method to prevent and treat Alzheimer's disease in a human subject comprising parenteral or oral administration of citrate to said subject.
